(19) United States
(12) Patent Application Publication
(10) Pub. No.: US 2011/0262518 A1
(43) Pub. Date: Oct. 27, 2011

(54) Treatment for cardiac injuries created by myocardial infarction

(76) Inventors: Stuart K.J. Smyth, Shelburne, VT (US); Jeanne M. Lesniak, Marco Island, FL (US)

(21) Appl. No.: 12/799,507

(22) Filed: Apr. 27, 2010

(51) Int. Cl. A61K 9/14; A61B 17/34; A61K 9/00; A61P 9/10; A61K 38/22; A61K 38/16; A61K 38/21; A61K 35/12; A61M 25/00; A61K 38/18

(52) U.S. Cl. 424/423; 604/523; 604/264; 424/426; 424/85.4; 424/93.7; 514/8.1; 514/17.2; 514/8.5; 514/8.9; 514/8.7; 514/8.4; 514/9.1

(57) ABSTRACT

An injectable, high potency (high capillary force) composite comprised of bioceramic spheres having a smooth porous macroarchitecture and porous microarchitecture with interconnected pores and may be combined with various electrospun polymer fibers, thus achieving a biphasic composite implant device whereby a primary phase stabilizes, reinforces, restricts and constricts the expansion of dysfunctional and diseased cardiac muscle tissue, and a secondary phase providing a matrix structure within the bioceramic composite for the regeneration of dysfunctional and diseased cardiac tissue; an injectable composite implant material containing pharmaceutical agents or may contain stem cells, and other DNA materials; an injectable, high potency, bioceramic composite material providing a specific means to alter cardiac muscle geometry so as to normalize cardiac wall stress, aid in the reduction of local stresses in the border zone or in the actual infarct as well as multiple peri-infarct border zone modifications that have been implicated in pathological remodeling.

This invention relates to the treatment of cardiac injuries created by myocardial infarction. This invention further relates to a method and means for the reinforcement and stabilization of injured cardiac tissues created by myocardial infarction. More specifically, this invention relates to an injectable, therapeutic, high potency, microporous bioceramic tissue reinforcement and cardiac muscle tissue stabilization agent that normalizes cardiac muscle stress and reduces local elevated stresses in the border zone and actual infarct, as well as the global effect of multiple peri-infarct border zone modifications that have been implicated in pathological remodeling.

### BACKGROUND TO THE INVENTION

Myocardial infarction, commonly known as a heart attack, occurs when the blood supply to part of the heart is interrupted causing some heart cells to die. This is most commonly due to occlusion of a coronary artery following the rupture of a vulnerable atherosclerotic plaque, which is an unstable collection of lipids and white blood cells in the wall of the artery. The resulting ischemia and oxygen shortage, if left untreated for a sufficient period of time, can cause damage and or death of heart muscle tissue.

If impaired blood flow to the heart lasts long enough, it triggers a process called the ischemic cascade; the heart cells in the territory of the occluded coronary artery die (chiefly through necrosis) and do not grow back. A collagen scar forms in its place. Recent studies indicate that another form of cell death called apoptosis also plays a role in the process of tissue damage subsequent to myocardial infarction. As a result, the individual's heart will be permanently damaged. This scar tissue also puts the patient at risk for potentially life-threatening arrhythmias, and may result in the formation of a ventricular aneurysm that can rupture with grave consequences.

Injured heart tissue conducts electrical impulses more slowly than normal heart tissue. The difference in conduction velocity between injured and uninjured tissue can trigger re-entry or a feedback loop that is believed to be the cause of lethal arrhythmias. The most serious of these arrhythmias is ventricular fibrillation, an extremely quick and chaotic heart rhythm that is the leading cause of sudden cardiac death. However, ventricular tachycardia typically results in rapid heart rates that prevent the heart from pumping blood effectively. Cardiac output and blood pressure may fall to dangerous levels, which can lead to coronary ischemia and extension of the infarct.

Complications may occur immediately following a myocardial infarction (in the acute phase), or may need time to develop (a chronic problem). After an infarction, an obvious complication is a second infarction, which may occur in the domain of another atherosclerotic coronary artery or in the same zone if there are any live cells left in the infarct. In terms of congestive heart failure (CHF) there are over 500,000 new cases diagnosed each year in the USA and over 5 million Americans suffer from CHF. The costs associated with CHF in the USA are in excess of $29 billion annually. With an increasingly aging population, the problems associated with CHF are expected to grow in the next coming decade. One of the leading causes of CHF is acute myocardial infarction. A myocardial infarction may compromise the function of the heart as a pump of circulation, a state called heart failure. There are different types of heart failure; left-sided or right-sided (or bilateral) heart failure may occur depending on the affected part of the heart, and its low-output type of failure. If one of the heart valves is affected, this may in fact cause dysfunction, such as mitral regurgitation in the case of the left-sided coronary occlusion. The incidence of heart failure is particularly high in patients with diabetes and requires special management strategies.

Myocardial rupture is most common three to five days after myocardial infarction, commonly of small degree, but may occur one day to three weeks later. In the modern era of early revascularization and intensive pharmacotherapy as treatment for MI, the incidence of myocardial rupture is about 1% of all MI's. This may occur in the free walls of the ventricles, the septum between them, the papillary muscles, or less commonly the atria. Rupture occurs because of increased pressure against the weakened walls of the heart chambers due to a heart muscle that cannot pump blood out effectively.

A heart attack is indeed a medical emergency which demands immediate attention and activation of emergency services. The ultimate goal of the management in the acute phase of the disease is to salvage as much myocardium as possible and prevent further complications. With the passage of time, the risk of damage to the heart muscle increases. Oxygen, aspirin, nitroglycerin and analgesia are usually administered as soon as possible. Other methods to treat a patient include standard emergency services via trained paramedic personnel, Reperfusion, Thrombolytic therapy, percutaneous coronary intervention, Coronary artery bypass surgery, monitoring of arrhythmias and rehabilitation. There are several new therapies under investigation. Patients who receive stem cell treatment by coronary artery injections of stem cells derived from their own bone marrow after myocardial infarction have shown improvements in left ventricular ejection fraction and end-diastolic volume not seen with placebo. Clinical trials of progenitor cell infusion as a treatment approach to ST elevation MI are proceeding.

Currently there are 3 biomaterial and tissue engineering approaches for the treatment of myocardial infarction. These three approaches are in their early stages of medical research and development. The first involves polymeric left ventricle restraints in the prevention of heart failure. The second utilizes the in vitro engineered cardiac tissue, which is subsequently implanted in vivo. The final approach entails injecting cells into the myocardium to create in situ cardiac tissue. Indeed, one of the most actively pursued approaches to treating CHF associated with acute myocardial infarction is cellular transportation into the infarct border zone region to improve regional and global pump function. A variety of different types of cells have been injected into the injured myocardium. For example, cells such as myoblasts, endothelial precursor cells, embryonic stem cells and bone marrow stromal cells. Furthermore, in addition to cells, some scientists have also included extracellular matrix materials with or without cells. The fact remains that most of the research and clinical studies to date have produced mixed results. For example, survival and engraftment of the implanted cells are poor, conclusive myocyte regeneration remains undemonstrated, and yet most cellular and/or extra cellular matrix material injection approaches are still able to reduce the loss of function after an infarct event. Debates continue as to how the addition of cells and/or synthetic extracellular matrix materials mitigates function loss as quantified by metrics such as injection fraction. Even though the injection of such cells and materials to the injury may be beneficial in many ways, such as through protective angiogenesis and cytokine-mediated reduction in apoptosis, one particular interesting benefit of these treatments can be the simple change to the ventricular-geometry and mechanics leading to a reduction of local elevated stresses that have been implicated in pathological remodeling. Early studies have indicated that even small fractional changes in left ventricle wall volume may significantly alter cardiac mechanics when properly situated. More precisely, research has been focused upon the effect of injecting noncontractile biomaterials into the border zone and actual infarct, indeed the two most common locations of therapy, as well as the overall or global effect of multiple peri-infarct border zone modifications.

Ceramic material, with it biocompatibility and resistance to wear, is ideally suited for a whole variety of medical implant applications, from artificial joints to implantable electronic sensors, stent technology, stimulators and drug delivery devices. For well over two decades, alumina, zirconia, hydroxyapatite and other ceramics have successfully proven their ability to withstand the harsh environment of the human body. In some instances, ceramics may be preferable to that of polymers because some polymer implant coatings, such as those utilized for drug elution, may be attributed to inherent pronin-flammatory effects. In an article published in Catheter Cardiovascular Interv. 2003 November; 60 (3): 408-409, it was reported that “several polymer coatings have shown an induction of inflammatory response and increased neointima formation”. The effect of a new inorganic ceramic nanoporous aluminum oxide coating on neointima proliferation and its suitability as a carrier for the immunosuppressive drug tacrolimus was investigated. The conclusion from the study was that the ceramic coating of coronary stents with a nanoporous layer of aluminum oxide in combination with tacrolimus resulted in a significant reduction in neointima formation and inflammatory response.

The use of bioceramics in human implant surgery began in the 1970's when the first generation of alumina products demonstrated superior wear, compared to the traditional metal and polyethylene materials. Traditional metal-polyethylene hip system wear generates polyethylene particulate debris, inducing osteolysis, weakening of surrounding bone and results in loosening of the implant, a primary cause of costly revision operations. Ceramic materials generate significantly less polyethylene debris when used in conjunction with polyethylene acetabular components in bearing couples. Indeed, state-of-the-art ceramic-on-ceramic technology, where an alumina femoral head is mated with an alumina acetabular cup, totally eliminates polyethylene debris and reduces wear significantly. Advances in material quality and processing techniques and a better understanding of ceramic design led to the introduction of second generation alumina components in the 1980's that offered even better wear performance.

In the 1980's hydroxyapatite coated hip replacements were being used to provide fixation, rather than bone cement, and the first human implantations for bone repair. A large research and development industry subsequently grew up and a variety of materials is now available. In terms of coatings, the application of hydroxyapatite to the surface of an implant component is important in determining its properties. There are a range of techniques for applying bioceramic to implants including plasma spraying, and some can now be performed in a controlled way while retaining the chemical nature of the bioceramic coating material. Some of the methods have the additional benefit of being able to produce nano-thin layers at low temperatures, thus preventing phase change. Incorporation of other bioactive molecules into these coatings is possible and may enable enhanced function.

Hydroxyapatite (HAP) is a form of calcium phosphate. Hydroxyapatite is chemically similar to the mineral component of bones and hard tissue in mammals. It is one of the few materials that are classified as bioactive, meaning that it will support bone ingrowth and osteointegration when used in orthopedic, dental and maxillofacial applications. Incorporation of active agents or drugs by physical absorption within porous HA-based implants has been frequently reported for orthopedic uses. Very recently, there has been growing interest in nanocrystals of HAP as carriers for bioactive agents. HAP has shown as a potential carrier for gene. The National Research Development Corporation of India has signed a license Agreement with American Bioscience to develop a calcium phosphate nanotechnology for non-viral gene delivery.

WO 2002/255144, WO 2004/02420, JP 2003/342113, WE 2003/009762 and WE 6426114 demonstrate that a great deal of research has also been conducted on various methods of producing hydroxyapatite coatings on metallic substrate surfaces for medical devices. Alternative examples to that of hydroxyapatite would be inorganic biomaterial such as bioglass, calcium oxide and phosphorous oxide. Indeed, bioglass, including glass and glass-ceramics, is currently used as implant materials.

A variety of ceramics and delivery systems have been used to deliver chemicals, biological and drugs at various rates for desired periods of time from different sites of implantation. In vitro and in vivo studies have shown that ceramics can successfully be used as drug-delivery devices. Matrices, inserts, reservoirs, cements and particles have been used to deliver a large variety of therapeutic agents such as antibiotics, anticancer drugs, anticoagulants, analgesics, growth factors, hormones, steroids and vaccines.

Medical implants delivering drugs are often used to ensure efficient medication at body sites where systemic administration is insufficient or could be harmful. The combination of a drug delivery coating on a mechanically supporting substrate is a beneficial concept for implants exposed to loads. Examples of these are both stents and orthopedic implants. Many of the commercially available coated stent implants are designed to release drugs locally and with a predefined rate of a polymer matrix. The-polymers used are either inert, bio-erodible or biodegradable. In spite of their successful release capability, they have often failed in regards to biocompatibility, long-term chemical and mechanical stability.

One important aspect in research into ceramics or other targeted drug carriers and slow drug release is preventing the risk of so-called “dose dumping”. Dose dumping can occur if the carrier based structure releases the whole dose within a short period of time instead of as a slow, sustained release.

Using ceramic materials containing pharmaceuticals makes possible the controlled release of active components. Ceramic materials can be tailored for different types of pharmaceuticals, thereby targeting different clinical needs. Research continues to be focused upon the correct particle size, porosity and surface structure. These characteristics affect how a drug will be released from the carrier into the targeted area within the body. The use of slow-release carriers can, therefore minimize the risk of overdosing since the drug will be released in a controlled way rather than all at once. However, research is ongoing in order to find the optimum carrier for the slow-release of drugs and other fluids in order to prevent dose dumping and to increase the quantity of drugs to be confined within a carrier. Evidence of dose dumping has been observed when intake of a drug has been combined with the consumption of alcohol. If this occurs when, for example, a ceramic carrier in the form of a drug pellet is loaded with a highly potent drug, severe or lethal side effects may result. Indeed, research on how to avoid this in the new class of pellets is therefore essential if constant drug release kinetics is to be achieved during transit through the body. Correct release of the drug in the intestinal system is therefore paramount.

Ceramics in a number of compositions or forms are widely used in biomaterials and devices. Alumina, Zirconia, Hydroxyapatite, Mullite, Silica and Titinia are examples of ceramics utilized in implants or prosthetic devices. Other suitable ceramic materials which are utilized or may be coated upon a primary ceramic structure or agent may be materials such as alumina, but are not limited to, calcium phosphate, calcium polyphosphate, tricalcium phosphate, dicalcium phosphate dehydrate, calcium hydrogen phosphate, hydroxyapatite, calcium metaphosphate, tetracalcium phosphates, heptacalcium decaphosphate, calcium pyrophosphate, monetite and octacalcium phosphate. However, beyond the ability to control porosity and solubility, the ideal ceramic structures or substances that may be bonded to an implant or substrate material must overcome their main drawback; which is their brittleness.

The development of a number of effective cardiovascular implants over the last 45 years has improved the quality and length of life of people in the vast majority of industrialized nations of the world where diseases of the heart and blood vessels remain the most common cause of death. Prosthetic heart valves and electronic pacemakers may be some of the most exciting and successful of the cardiovascular implants. A vast number of cardiovascular implants incorporate the usage of certain bioceramic materials so to aid the patient with cardiac function. The human heart beats approximately 35 million times a year to pump millions of gallons of blood through more than 12,000 miles of arteries. During the hearts non-stop activity, constant repair and replacement of tissue take place. Engineering a device or implant to produce either the function of the heart or aid in the reinforcement and stabilization of cardiac tissue leading to a reduction of local elevated stresses that have been implicated in pathological remodeling; and with the necessary confinement of therapeutic drugs to treat injured cardiac tissue, indeed presents a monumental challenge. The performance requirements of implantable cardiac biomaterials are most severe and must be nonthrombogenic, extremely durable, achieve the least possible disturbance and interference with the flow of blood and even capable of confining certain therapeutic pharmaceutical agents. The material utilized should not be unduly difficult to implant into the injured cardiac tissue, and even more specifically, without undue difficulty when implanting such biomaterials into the border zone and actual infarct, indeed the two most common locations of therapy, as well as the overall or global effect of multiple peri-infarct border zone modifications.

All myocardial infarctions go through physiologic changes, leading to progressive enlargement of the ventricle, severe depression of muscle cells and ventricular function, often leading to heart failure. Within seconds of a coronary artery occlusion, the process starts and changes occur in the ischemic tissue, which result in abnormal wall motion, high stresses, and wasted energy. Cardiac function is depressed, and when severe, cardiogenic shock occurs with increased morbidity and mortality.

Over the next few days, infarct extension and expansion occur. Infarct extension results in a further permanent loss of myocardial capacity due to inadequate blood flow to support the heart's workload. Infarct expansion is a fixed, permanent, disproportionate regional thinning and dilatation of the infarct zone due to slippage of tissue layers. This ventricular dilatation and wall thinning lead to very high stresses around and within the infarct tissue, which initiates ventricular remodeling and subsequent heart failure. In fact, within 5 years after a first heart attack, 28% of men and 37% of women develop chronic heart failure [AHA, Heart and Stroke Statistics 2009].

Thus, the infarct region presents a mechanical disadvantage to the heart, which accounts for the subsequent pathophysiological processes leading to heart failure.

Because definitive myocardial regeneration remains undemonstrated, there is a documented need for and, in fact, provide a more effective method and means of injectable treatment for injured cardiac tissue following an infarction. More specifically, to provide an extremely safe, bioinert, proven, extremely durable, noncontractile, non-brittle, non-migratory and non-antigenic biomaterial/agent in order to successfully treat dysfunctional or injured cardiac tissue created by myocardial infarction; and all by way of the possible injection of a high potency microporous bioceramic agent into the border zone and actual infarct, as well as the global effect of multiple peri-infarct border zone modifications in order to secure a reduction of local elevated stresses that have been implicated in pathological remodeling; and capable of confining therapeutic drugs to assist in the therapeutic treatment of the injured cardiac tissue. Implanting such a material or agent ought to effectively alter mechanisms of the ventricle and should normalize cardiac muscle stress in an injured ventricle by way of reinforcing, stabilizing and thus thickening cardiac tissue thus, at the very least, resulting in an increased reduction in remote and border zone stresses.

### OBJECTS OF THE INVENTION

It is therefore an object of the invention to provide an effective treatment of cardiac injuries following a myocardial infarction; a method and means to overcome the disadvantages or limitations of existing treatments or the current biomaterials. It is within the same object of the invention to provide for the reinforcement and stabilization of injured cardiac tissues which has been injured by myocardial infarction; and in the form of an injectable, high potency, microporous bioceramic tissue reinforcement and stabilization agent that normalizes cardiac wall stress and aids in a reduction of local elevated stresses in the border zone and actual infarct, as well as the global effect of multiple peri-infarct border zone modifications that may implicated in pathological remodeling; and it is in conjunction with the same object of the invention that the tissue agent include a high potency, porous microarchitecture which may bind cardiac muscle cells and tissue and may confine therapeutic drugs to treat injured cardiac tissue created by myocardial infarction.

### SUMMARY OF THE INVENTION

According to a first aspect of the invention there may be provided a high potency, durable, particulate, micro-porous bioceramic cardiac reinforcement and cardiac wall tissue stabilization agent, having interconnected pores, and which may provide a means to alter cardiac muscle geometry so as to normalize cardiac wall stress, and more specifically, may aid in the reduction of local stresses in the border zone, actual infarct, as well as multiple peri-infarct border zone modifications that may have been implicated in pathological remodeling. Further according to the invention the particles may be in the form of spheres.

Yet further according to the invention there may be a high potency, durable bioceramic cardiac reinforcement and cardiac wall tissue stabilization agent that may contribute to changes in cardiac mechanics; and more specifically may contribute to tissue rigidity or stiffness and overall stress response to strain of the border zone, actual infarct, as well as multiple peri-infarct border zone modifications that may have been implicated in pathological remodeling.

Further according to the invention the micro-pores may be exposed on the surface of the spheres, hence rendering the surface of the spheres smooth on a macro-scale, but may be porous and uneven on a micro-scale to provide a means to enhance and promote the formation of cardiac tissue and cardiomyocytes in the pores and the binding of cardiac tissue and cardiomyocytes to the spheres.

Further according to the invention the micro-pores may be exposed on the surface of the spheres, hence rendering the surface of the spheres smooth on a macro-scale, but may be porous and uneven on a micro-scale to provide a means to enhance and promote the permeation of cardiac tissue and cardiomyocytes, liquids and gases in the pores in order to provide mechanical stability within the cardiac tissue which may therefore normalize cardiac wall stress.

Further according to the invention the micro-pores may be exposed on the surface of the spheres, hence rendering the surface of the spheres smooth on a macro-scale, but may be porous and uneven on a micro-scale to provide a means to enhance and promote the permeation of cardiac tissue and cardiomyocytes, liquids and gases in the pores in order to reinforce and stabilize cardiac tissue and which thereby may result in a thickening and stiffening within the cardiac tissue which may therefore normalize cardiac wall stress.

Yet further according to the invention the micro-pores may be exposed on the surface of the spheres, hence rendering the surface of the spheres smooth on a macro-scale, but may be porous and uneven on a micro-scale to provide a means to enhance and promote the permeation of cardiomyocytes and tissue in the pores in order to reinforce and stabilize cardiac tissue and which thereby and may result in a thickening and stiffening within the cardiac wall tissue thus normalizing cardiac wall stress. Further according to the invention the reduction of cardiac stresses following a myocardial infarction may in turn minimize stress-induced apoptosis and border zone extension and expansion, reducing further remodeling and preventing the progression into congestive heart failure.

Yet further according to the invention the micro-pores may be exposed on the surface of the spheres, hence rendering the surface of the spheres smooth on a macro-scale, but may be porous and uneven on a micro-scale to enhance and promote the permeation of cardiomyocytes, and tissue in the pores in order to reinforce, stabilize and may thus result in a thickening and stiffening or the creation of a bulge in targeted cardiac tissue, such as the apical anterior wall, thus achieving a minimum wall volume increase of greater than, on average, 0.5%, and therefore demonstrate that even a small amount of resulting tissue volume increase or thickening and stiffening following the introduction of the reinforcement and stabilization of the tissues may alter cardiac mechanics and decrease wall stresses.

Yet further according to the invention, the pores on the surface of each sphere are connected to at least some of the pores inside such sphere via blow-holes and at least some of these internal pores are in turn interconnected, so that in addition to a high porosity, the spheres have a high permeability to tissue, cardiomyocytes, gas and liquids.

The durable particles may have a porosity of between 25% and 85% per volume.

The pores of the durable particles may have diameters in the range of from 0.3 to 15 micrometer.

The particles may have a diameter larger than 25 micrometer, typically 45 to 400 micrometer.

The particles may be mobilized by mixing the particles with a bio-suitable carrier to render the particles injectable into the injured cardiac tissue of a patient.

Further according to the invention, the diameter size fractions of the particles do not differ more than 20-100 micrometer from each other in order to restrict bridging of the particles in a hypodermic needle during injection under pressure into the injured cardiac tissue of a patient, in use.

The appropriate permanent ceramic material may be selected from the group consisting of durable sintered aluminum oxide (alumina); sintered zirconium oxide (zirconia); and the combination thereof; as well as oxides such as sapphire or ruby.

The appropriate ceramic material may be selected from Mullite, Silica and Titinia.

The appropriate ceramic material may be selected from Silicon Nitride, Silicon Carbide, Titanium Carbide, Titanium Nitride and Tantalum Carbide.

The appropriate biodegradable or resorbable ceramic material may be selected from Hydroxyapatite.

Further to the invention, the appropriate ceramic material may be selected from ceramics consisting of; bioactive glass, machineable and phosphate glass-ceramic, polycrystalline glass-ceramic, liquid-phase sintered ceramic, hot pressed ceramic or glass-ceramic, carbon-ceramic, zeolite, ceramic-polymer, Sol-gel glass or ceramic and multi-phase ceramics may be considered for usage.

The additional ceramic materials which may be contribute to the therapeutic treatment, but are not limited to; calcium phosphate, calcium polyphosphate, tricalcium phosphate, dicalcium phosphate dehydrate, calcium hydrogen phosphate, calcium metaphosphate, tetracalcium phosphates, heptacalcium decaphosphate, calcium pyrophosphate, monetite and octacalcium phosphate.

The porous, interconnected microarchitecture of the ceramic material may be coated and may incorporate diamond, carbon or metals.

The cardiac reinforcement and tissue stabilization agent may be in the form of a composite.

The cardiac reinforcement and tissue stabilization agent may be in the form of an injectable ceramic-polymer mesh composite.

According to the second aspect of the invention there is a high potency, durable bioceramic reinforcement and cardiac wall stabilization agent comprised of particles with a porous, interconnected microarchitecture which may provide a suitable means to confine variety of chemical and pharmaceutical agents and which may enhance the overall therapeutic function and outcome of the tissue reinforcement and stabilization agent, thus promoting rehabilitation of the injured cardiac tissue.

According to the third aspect of the invention, the ceramic materials may be used to form a composite which may include or incorporate biologically suitable organic or inorganic biomaterials in order to aid in achieving the therapeutic goal of changing the cardiac mechanics in order to effectively reduce pathological cardiac wall stresses; and may ultimately demonstrate improvement in global pump function; and which may include, but are not limited to: a variety of polymers, copolymers, hydrogels, adhesives, cellulose based biomaterials (e.g. microcrystalline cellulose), extracellular matrices, cellular engraftments, a variety of stem cells, stromal cells and bone cells. Further to the invention and according to the third aspect, the ceramic materials may be used to form a composite and may include the use of the following materials; keratin, silk, fibrin, thrombin, collagen, gelatin, alginic acid and salts, chitin, chitosan, hyaluron, hyaluronic acid, cellulose, n-acetyl glucosamine, proteoglycans, glycolic acid polymers, lactic acid polymers, glycolic acid/lactic acid co-polymers, fibrin cross-linker, calcium ions, sucrose, lactose, maltose, dextrose, mannose, trehalose, sorbitol, albumin, sorbate, polysorbate, sodium bicarbonate/citric acid, sodium bicarbonate/acetic acid, calcium carbonate/acetic acid, antibiotics, anticoagulants, steroids, cardiovascular drugs, alpha blockers, beta-blockers, growth factors, antibodies (poly and mono), chemoattractors, anesthetics, antiproliferatives/antitumor agents, antivirals, vaccines, cytokines, colony stimulating factors, antifungals, antiparasitics, antiinflammatories, peptides, antiseptics, hormones, vitamins, glycoproteins, fibronectin, proteins, carbohydrates, proteoglycans, antigens, nucleotides, lipids, liposomes, fibrinolysis inhibitors and gene therapy reagents.

The particles may be activated prior to injection by impregnation with a substance selected from the group consisting of traces of a bioactive resorbable bioceramic or other bioactive coating; as well as the serum from the blood of a patient.

The particles may be mobilized for injection by mixing the particles with a bio-suitable pharmaceutically acceptable carrier selected from the group consisting of a gel, and a viscous, lubricating liquid. According to a fourth aspect of the invention there is provided a device for reinforcement and stabilization within a portion of cardiac tissue in vivo, comprising an effective amount of cardiac tissue reinforcement and stabilization agent according to a first aspect of the invention; and applying means for applying the reinforcement and stabilization which may result in a thickening and stiffening of the injured cardiac tissue.

The applying means may be in the form of a needle placed through a catheter guide and having so as to deliver the bioceramic therapeutic tissue stabilization agent at the targeted site.

The cardiac tissue reinforcement and stabilization agent may be disposed inside the syringe or suitable injection device.

According to a fourth aspect of the invention there is provided use of a reinforcement and stabilization agent according to the first aspect of the invention in a method for treating injured cardiac tissue created by myocardial infarction in a patient.

According to a fifth aspect of the invention there is provided use of a reinforcement and wall stabilization agent according to the first aspect of the invention in the manufacture of a device for use in a method for treating injured cardiac tissue created by a myocardial infarction in a patient.

According to a sixth aspect of the invention there is provided a method of treating injured cardiac tissue created by a myocardial infarction in a patient including the step of locating an effective amount of reinforcement and cardiac wall thickening tissue stabilization agent according to the first aspect of the invention in an affected area of the patient in need thereof. The step of locating the tissue stabilization agent in the affected area may include the further step of injecting the cardiac tissue reinforcement and stabilization agent into the of the border zone, actual infarct, as well as multiple peri-infarct border zone modifications that may have been implicated in pathological remodeling.

The method may include the further step of moistening the cardiac tissue stabilization agent with blood drawn from the patient into a syringe containing the agent, such that the red blood cells accumulate on the surface of the particles and the serum is drawn into and stored inside the particles.

The method may include the further step of activating the particles prior to injection by impregnation with a substance selected from the group consisting of traces of a compatible bioactive resorbable biomaterial, and serum from the blood of a patient in order to achieve the desired outcome.

The method may include the further step of mobilizing the particles for injection by mixing the particles with a bio-suitable pharmaceutically acceptable carrier selected from the group consisting of a gel, and a viscous, lubricating liquid.

According to a seventh aspect of the invention there is provided a method of reinforcing and stabilizing a portion within cardiac tissue of a human body including the step of introducing to that portion a volume of the issue stabilization agent according to a first aspect of the invention.

The method may include, but not limited to, the further step of injecting the tissue stabilization agent through the femoral artery and inserted into the Left Ventricle retrograde through the aortic valve.

Alternatively, the method may include the further step of injecting the tissue reinforcement and stabilization agent into the cardiac tissue itself via an open heart procedure or minimally invasive procedure.

The invention may now be described further by way of a non-limiting example of a preferred embodiment of the invention.

The cardiac reinforcement and tissue stabilization agent according to the present invention may be prepared, according to the type of ceramic selected; for example, with what may include a method including the steps of: milling solid ceramic into powder (1 micrometer diameter particles); adding a combustible substance known in the trade of manufacturing porous ceramic structures to the powder;—mixing the powder with water to form a paste or slurry; forming solid spherical particles from the slurry by applying methods known in the art; sintering the spherical particles at a temperature of 1350 degrees C. to form inert micro-porous ceramic spheres having pore sizes of between 0.3 to 15 micrometers and diameter of between 25 to 400 micrometers; and—screening the spheres into pre-selected size ranges, with the respective diameters of spheres in a particular range not differing more than 20-100 micrometer from each other.

The applicants have found that a cardiac tissue reinforcement and stabilization agent according to the present invention may meet some of the critical clinical requirements for the successful treatment of injured cardiac tissues created by myocardial infarction, inter alia:
may be immobile, reinforcement and tissue stabilization agent and thus may result in a strengthening, thickening and stiffening of cardiac wall tissue;
may conform to physical requirements for placement by injection;
may be non-immunogenic, hypoallergenic and non-antigenic;
may be bio-compatible, with virtually no measurable or minimal inflammatory response;
may result in sustainable tissue thickening and stiffening
may provide satisfactory and rapid tissue ingrowth and thus result in tissue thickening in the applied area with adequate or minimal fibrotic ingrowths;—retains tissue stabilization/enhancement and in some cases replacement over prolonged periods of time;
when injected into the of patients suffering a myocardial infarction or congestive heart failure the appropriate volume of ceramic spheres reinforce, stabilize and thus result in a thickening and stiffening of the border zone, actual infarct, as well as multiple peri-infarct border zone modifications that may have been implicated in pathological remodeling;
includes spheres having particle sizes above 25 microns and surface roughness or pores exposed on the surface that instigate adhesion to host tissue; and
provides synthesis in functionality between a carrier and the injectable composite material.

In use, the tissue reinforcement and stabilization agent may be disposed inside a syringe having an outlet and needle attached to the outlet with a bore size adequate for the infusion of the injectable bioceramic tissue reinforcement and stabilization agent. The stabilization agent may, but not necessarily so, be then activated by impregnating the spheres with traces of a bioactive resorbable bioceramic or other complimentary therapeutic biomaterials to treat the targeted area, or aid in the overall function of providing adequate tissue rigidity or stiffness. The spheres may be mobilized for injection by mixing the spheres with a bio-suitable carrier gel or a viscous, lubricating carrier liquid. Thereafter, the activated and mobilized tissue stabilization agent is injected into the tissue in the area to be treated, reinforced thickened or stabilized, such as the injured cardiac tissue created by a myocardial infarction.

The spheres could alternatively be activated and mobilized by the in situ impregnation with serum from the blood of a patient. This includes the moistening of the tissue stabilization agent with blood drawn from the patient into the syringe containing the tissue stabilization, such that the red blood cells accumulate on the surface of the spheres and the serum is drawn into and stored inside the spheres. The applicants foresee that this may stimulate tissue forming on the surface even more and may reduce any immunogenic reactions to the tissue stabilization agent even further.

The applicants further foresee that the tissue stabilization agent according to the invention could be manufactured and prepared for application at a relatively low cost.

It will be appreciated that the application of the tissue stabilization agent according to the present invention is not limited to use in the treatment of injured cardiac tissue created by myocardial infarction, but could be applied toward other vascular conditions where required, such as mitigation of mitral valve insufficiency. The characteristics of the tissue stabilization agent of the present invention, such as, inter alia, the low cost of manufacture; the safety; stability; immobility after application; and the ease of application thereof, not only may overcome some the disadvantages of the prior art, but also renders the tissue reinforcement and stabilization agent and the method of the invention suitable and available to a wide range of patients including those suffering from vascular related disorders.

The description of the embodiments of the present invention is given above for the understanding of the present invention. It will be appreciated further that the invention is not limited to the particular embodiments described herein, but is capable of various modifications and rearrangements as will be apparent to those skilled in the art without departing from the scope of the appended claims.

### DETAILED DESCRIPTION OF THE DRAWINGS

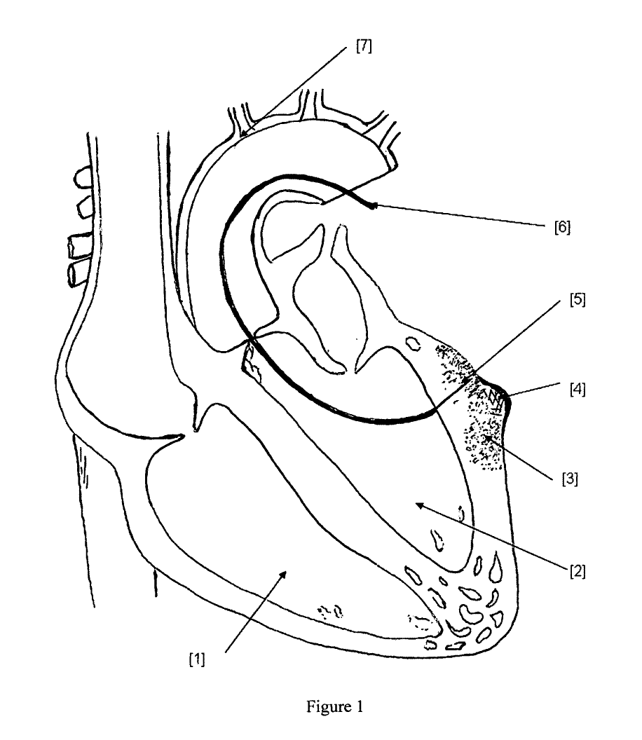

FIG. 1 shows the device being introduced past the aortic valve [7] via the percutaneous catheter [6] through into the left ventricle [2], adjacent to the right ventricle [1], and the needle being introduced into the cardiac muscle of the heart at the border zone of the infarct [4], whereupon the injectable composite [3] is injected to begin the treatment.

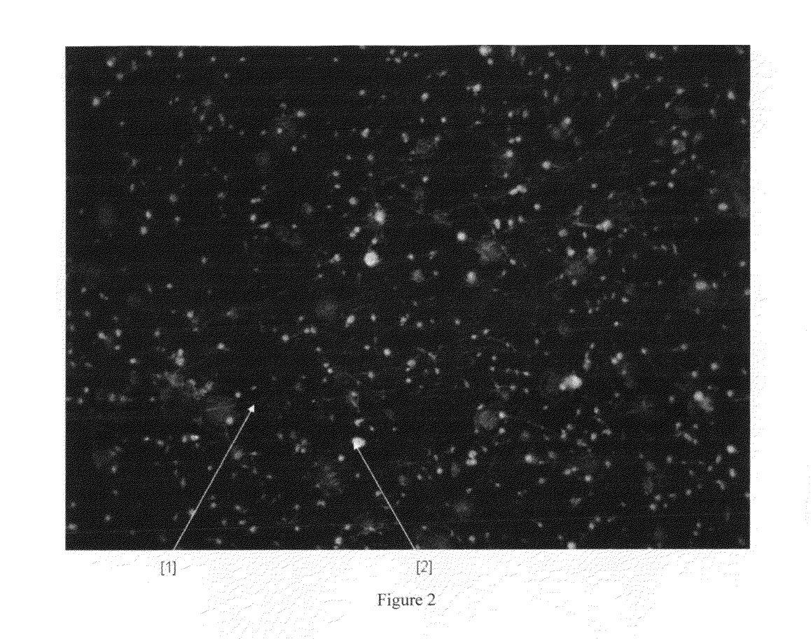

FIG. 2 shows the composite device being comprised of electrospun PCL or polymer fibers [1] when combined with the microporous bioceramic spheres [2].

1. An injectable, biphasic, high-capillary force ceramic implant composition for the stabilization and restriction of cardiac muscle stress and local elevated stresses in the border zone, actual infarct and multiple peri-infarct border zone modifications, injected following a myocardial infarction, and with the composition restricting and constricting the global expansion of diseased cardiac muscle tissue following a myocardial infarction and providing a means for the regeneration of new cardiac tissue, and comprised of:
a plurality of bioceramic particles in a carrier gel, wherein each particle is microporous having fully interconnected pores throughout, and the particles are in the form of smooth spheres exposed on the surface at the macro-scale, and the pores on the smooth surface are connected to at least some of the pores inside the spheres via blow-holes and the internal pores are in turn interconnected rendering the spheres highly porous with a high permeability to tissue, cells, gases and liquids.

2. An injectable, biphasic, high-capillary force ceramic implant according to claim 1 wherein the primary phase of the biphasic implant is to immediately stabilize, restrict and constrict the expansion of dysfunctional and diseased cardiac muscle tissue thereby preventing or reducing further damage to the cardiac muscle due to heart failure following a myocardial infarction.

3. An injectable, biphasic high-capillary force ceramic implant according to claims 1 and 2 wherein the biphasic bioceramic implant composition is comprised of microporous bioceramic spheres having fully interconnected pores and whereby the high-capillary force (high potency or high absorptive force) spheres rapidly suction/absorb and draw tissue, cells, gas and liquids toward, adjacent to, within and throughout both the macroarchitecture and microarchitecture of the spheres thus providing the immediate bonding/restrictive and constrictive action and force during the primary phase of the implants introduction within the cardiac muscle tissue.

4. An injectable, biphasic, high-capillary force ceramic implant for the treatment of dysfunctional and diseased cardiac tissue according to claims 2 and 3 with a high-capillary force (high absorptive force) of between 55 kPa and 935 KPa creates an immediate suctioning force once injected and thus upon the cardiac tissue, cells, gases and liquids resulting in the immediate stabilization and restriction of the dysfunctional or diseased tissue and resulting in the encapsulation of both the dysfunctional and diseased cardiac tissue, as well as healthy cardiac tissue; and more specifically, stabilization, restriction and encapsulation of tissue at the border zone, peri-infarct region and the actual infarct region during the primary phase of the implants initial introduction into the cardiac muscle tissue.

5. An injectable, biphasic, high-capillary force ceramic implant according to claims 1 and 2 wherein the secondary phase of the biphasic implant provides a microporous, interconnected, ceramic scaffolding matrix structure for the regeneration of dysfunctional and diseased cardiac tissue, as well as for the seeding of new cells and tissue.

6. The composition according to claim 1, 2, 3, 4 wherein the spheres have a porosity of 25% to 85% per volume and the pores of the spheres have diameters in the range of 0.3 to 15 micrometer.

7. A composition according to claim 1, 2, 3, 4, 6 wherein the spheres have a diameter larger than 25 micrometer.

8. A composition according to claim 1, 3, 6, 7 wherein the particles have a diameter of 25 micron to 400 micrometer.

9. A composition according to any one of the preceding claims wherein the bioinert ceramic material is selected from a group consisting of aluminum oxide (alumina), zirconium oxide (zirconia); and the combination thereof; as well as the oxides sapphire or ruby.

10. A composition according to any one of the preceding claims wherein the material may be selected from Mullite, Silica, Titania, Silicon Nitride, Silicon Carbide, Titanium Carbide, Titanium Nitride and Tantalum Carbide, bioactive glass, machineable and phosphate glass-ceramic, polycrystalline glass-ceramic, liquid sintered ceramic, hot pressed ceramic, carbon-ceramic, ceramic polymer, Sol-gel glass, multi-phase ceramics.

11. A composition according to claims 1-8, wherein the appropriate biodegradable or resorbable ceramic material is, but is not limited to, hydroxyapatite.

12. A composition according to claims 1,5,10 wherein either the primary phase and the secondary phase of the biphasic ceramic composition will be combined with three dimensional polymer electrospun porous synthetic fibers comprised form a selection of polymers, but not limited to, polycaprolactone (PCL), poly (lactide-co-glycolide) (PGLA), poly (lactide-co-caprolactone) (PLCL), polylactide (PLA), polylactic acid-glycolic acid), poly (lactic acid), poly (glycolic acid), poly (orthoester), poly (phosphazene), a polymide, a polysaccharide, polydioxanone, polyanhydride, trimethylene carbonate, poly (b-hydroxybutryrate), poly (g-ethyl glutamate), poly (DTH iminocarbonate), poly(bisphenol A iminiocarbonate), polycynaoacrylate, Nylon, PEO, PEG, PBT, DT, DTE, DTE-co-DT, b-CDs, PPG, PTMO, PSX and polyphophosphazene.

13. A composition according to claims 1,5,10 wherein either the primary and the secondary phase of the biphasic ceramic composition will be combined with biodegradable electrospun fibers comprised of, but not limited to, modified polysaccharides (cellulose, microcrystalline cellulose, chitin, and dextran), gelatin, silk, hyaluronan, modified proteins (fibrin, casein) and a collagen.

14. A composition according to claim 1 wherein the spheres are mobilized for injection by mixing them with a pharmaceutical grade gel carrier and a viscous, lubricating liquid and a thermosensitive cross-linked polymer gel carrier.

15. A composition according to claims 9-14 wherein the composition of the biphasic implant is in a form of an injectable composite depending on the ratios of the composite.

16. A composition according to claims 1-5, 9-13 further comprising additional components which may be contained in either the primary phase and the secondary phase of the biphasic implant and selected from the group consisting of growth factors, chemotherapeutic agents, pharmacologic agents and biologically active agents such as antibiotics selected from the group consisting of aminoglycosides, carbacephems, carbapenems, cephalosporins, glycopeptides, marolides, monobactams, penicillin's, polypeptides, sulfonamides and tetracylides soluble in water and soluble in organic solvents, and particulate/colloidal silver or bismuth thiols; growth factors and biologically active agents selected from the group consisting of epidermal growth factor (EGF), transforming growth factor-alpha (TGF-[alpha]), transforming growth factor-beta (TGF-[beta]), human endothelial cell growth factor (ECGF), granulocyte macrophage colony stimulating factor (GM-CSF), nerve growth factor (NGF), vascular endothelial growth factor (VEGF), fibroblast growth factor (FGF), insulin-like growth factor (IGF), and/or platelet derived growth factor (PDGF); therapeutics selected from the group consisting of cytotoxins, antibodies, analgesics, anticoagulants, anti-inflammatory compounds, antimicrobial compositions, cytokines, interferons, hormones, lipids, oligonucleotides, polymers, polysaccharides, polypeptides, protease inhibitors, vasoconstrictors vasodilators, vitamins and minerals, vasoactive agents, neuroactive agents, anesthetics, muscle relaxants, steroids, anticoagulants, anti-inflammatory agents, anti-proliferating agents, anti-ulcer agents, antivirals, vaccine materials, immuno-modulating agents, cytotoxic agents, prophylactic agents, antigens, antibodies, fibrinogen, thrombin, plasticizer, fibronectin, cellular associated proteins and plasma derived proteins, Factor XIII, proteases, protease inhibitors or mixtures thereof.

17. A composition according to claims 5, 12, 13, 14 wherein the secondary phase of the biphasic implant composition provides the scaffolding or matrice structure for the seeding of stem cells, myoblasts, endothelial precursor cells, bone marrow stomal cells and extracellular matrix materials.

18. A method of claim 1-2 for the stabilization and restriction of cardiac wall stress and local elevated stresses in the border zone, actual infarct and multiple peri-infarct border zone modifications, injected following a myocardial infarction, and with the composition restricting and constricting the global expansion of dysfunctional and diseased cardiac muscle tissue following a myocardial infarction as well as providing a means for the regeneration of new cardiac tissue.

19. A method of claim 3-4 within cardiac wall tissue immediately following a myocardial infarction for the immediate bonding/restrictive and constrictive action and force during the primary phase of the implants introduction within the cardiac wall muscle tissue as well as the ensuing encapsulation of dysfunctional and diseased cardiac tissue, as well as healthy cardiac tissue; and more specifically, the stabilization, restriction and encapsulation of tissue at the border zone, peri-infarct region and the actual infarct region during the primary phase of the implants introduction into the cardiac muscle tissue.

20. A method of claim 5 wherein the secondary phase of the biphasic implant provides the microporous, interconnected, ceramic scaffolding matrix structure for the regeneration of diseased cardiac tissue, as well as for the seeding of new cells and tissue.

21. A method of claim 15 wherein the injectable biphasic bioceramic implant is in a form of an injectable composite depending on the ratios of the components.

22. A device according to claim 1-5 for the stabilization and restriction of cardiac muscle stress and local elevated stresses in the border zone, actual infarct and multiple peri-infarct border zone modifications, injected following a myocardial infarction, and with the composition restricting and constricting the global expansion of dysfunctional and diseased cardiac muscle tissue following a myocardial infarction and providing a means for the regeneration of new cardiac tissue.

23. A device according to claim 22 wherein the applying means is in the form of a catheter mechanism to be injected percutaneously into the wall of the cardiac muscle tissue at the site of the actual infarct including the peri-infarct region.

24. A device according to claim 22 wherein the applying means may be disposed of in a hypodermic needle-like/trocar-like mechanism and applied via open surgical technique into the wall of the cardiac muscle tissue at the site of the actual infarct as well as the peri-infarct region.

25. Use of an injectable, biphasic, high-capillary force ceramic implant composition for the stabilization and restriction of cardiac muscle stress and local elevated stresses in the border zone, actual infarct and multiple peri-infarct border zone modifications, injected following a myocardial infarction, and with the composition restricting and constricting the global expansion of dysfunctional and diseased cardiac muscle tissue following a myocardial infarction and as a means of providing for the regeneration of new cardiac cells and tissue.

26. Use of an injectable, biphasic, high-capillary force ceramic implant introduced percutaneously via a catheter mechanism through the femoral artery and inserted into the Left Ventricle retrograde through the aortic valve.
